(19) United States
(12) Patent Application Publication
(10) Pub. No.: US 2024/0095889 A1
(43) Pub. Date: Mar. 21, 2024

(54) SYSTEMS AND METHODS FOR MAGNETIC RESONANCE IMAGE RECONSTRUCTION WITH DENOISING

(71) Applicant: THE UNIVERSITY OF HONG KONG, Hong Kong (CN)

(72) Inventors: Ed Xuekui WU, Hong Kong (CN); Linshan XIE, Hong Kong (CN); Jiahao HU, Hong Kong (CN); Yujiao ZHAO, Hong Kong (CN); Christopher MAN, Hong Kong (CN)

(73) Assignee: THE UNIVERSITY OF HONG KONG, Hong Kong (CN)

(21) Appl. No.: 18/546,497

(22) PCT Filed: Feb. 25, 2022
(86) PCT No.: PCT/CN2022/077989

§ 371 Date: Aug. 15, 2023

Related U.S. Application Data
(60) Provisional application No. 63/156,225, filed on Mar. 3, 2021.

(51) Int. Cl. G06T 5/00; G06T 7/00; G06T 11/00

(52) U.S. Cl. CPC G06T 5/002; G06T 7/0002; G06T 11/006; G06T 2207/20081; G06T 2207/30168; G06T 2210/41; G06T 2211/441

(57) ABSTRACT

Systems and methods for improving magnetic resonance imaging relate to reconstructing multi-slice images based on sharing the strong structural similarities between adjacent image slices. In addition, a joint denoising method exploits these structural similarities. In part the reconstruction is based on use of a residual neural networks and denoising is achieved with a deep learning based strategy. The system and method have proved useful in both simulation and in vivo brain experiments, demonstrating significant noise reduction in all images and revealing more microstructural details in quantitative diffusion maps.

This application is a U.S. National Stage Application under 35 U.S.C. § 371 of International Patent Application No. PCT/CN2022/077989, filed Feb. 25, 2022, and claims the benefit of priority under 35 U.S.C. Section 119(e) of U.S. Provisional Patent Application No. 63/156,225 filed on Mar. 3, 2021, the entire contents of both of which are incorporated by reference for all purposes.

### FIELD OF THE INVENTION

The present invention relates to systems and methods for improving magnetic resonance imaging, and more particularly to reconstructing multi-slice and multi-contrast images and removing noise from those images.

### BACKGROUND OF THE INVENTION

Diffusion MRI offers a powerful approach to map tissue microstructure. However, it intrinsically suffers from a low signal-to-noise ratio (SNR), especially when spatial resolution or b-value is high. A typical diffusion MRI session produces image sets with same geometries but different diffusion directions and b-values. These diffusion-weighted (DW) images often share strong structural similarities despite their contrast differences.

Partial Fourier (PF) magnetic resonance imaging (MRI) has received abundant attention due to its potential in reducing the echo time or scan time. [1] A limited symmetrically sampled central k-space region will undermine the phase estimation accuracy, leading to degraded performance of traditional PF reconstruction, such as the projection-onto-convex-sets (POCS) method. [2] In recent years, multi-slice MR reconstruction has demonstrated its great potential in exploiting the similarities in image contents and coil sensitivity maps in adjacent slices. [3],[4] The image phase across adjacent slices should also be similar due to the slow variation of the main magnetic field and coil sensitivity maps. The multi-slice nature allows for adjacent slices with different sampling patterns, providing complementary information across different slices. [3]

Routine clinical MRI sessions acquire multi-contrast images with identical geometries to maximize diagnostic information, yet result in prolonged scan times. Images of different contrasts are often independently reconstructed despite their intrinsic anatomical similarities. The redundancy on the shared anatomical information can be utilized by jointly reconstructing multi-contrast images. Recent publications have demonstrated the benefit of jointly reconstructing images from MR data with multiple contrasts using deep learning (DL) to take advantage of the redundancy. [15]

Conventional parallel imaging uniformly under samples the k-space, which results in aliasing that manifests as coherent replicas of the original image content. Additional calibration data is required to assist in unfolding the aliasing. Incoherency across different contrasts can be introduced by orthogonally under sampling MR data of different contrasts, and exploited by jointly reconstructing multiple contrast MR data via low rank matrix completion [16].

Multi-contrast MRI is a useful technique to aid clinical diagnosis. It offers multi-contrast images with complementary diagnostic information. Although the signal intensity varies dramatically across different contrasts, the multi-contrast images often share highly correlated structure information from slice to slice because the neighboring sections of the underlying tissue often share strong structural similarities or correlations.

Most existing MRI denoising methods only carry out denoising on single contrast slices without using analogous structural information to support the restoration, i.e., existing single-contrast MRI denoising methods neglect the analogous structure information. Moreover, conventional deep learning (DL) based methods train a model for blindly denoising images with different noise levels, which compromises the performance. Alternatively, the trained model is trained for a specific level of noise reduction, which means multiple models are needed for different noise levels. A single model that can be adjusted to fit images with varying noise levels can offer better performance and higher flexibility.

### SUMMARY OF THE INVENTION

In one embodiment the present invention is a new method for jointly denoising diffusion-weighted (DW) images using low-rank matrix approximation. It exploits structural similarities of DW images, leading to significant noise reduction in all DW images and revealing more microstructural details in quantitative diffusion maps. With this method, similar patches from a DW image set are extracted to form low-rank patch matrices. A low-rank matrix approximation method is then applied to estimate noise-free patch matrices. The method has been evaluated for use with in vivo brain DW images.

The low-rank based method comprises the steps of (a): extracting reference patches using a sliding window, (b) searching for similar patches through block matching for each reference patch, (c) stretching similar patches to vectors and stacking them into a matrix to form a low-rank patch matrix, (d) estimating a noise-free patch matrix for each patch matrix through a weighted nuclear norm minimization (WNNM) model, and (e) converting estimated patch matrices back to images.

In a second embodiment the present invention is a residual network based reconstruction method that is provided for multi-slice partial Fourier acquisition, where adjacent slices are sampled in a complementary way. The anatomical structure and phase similarity of multi-slice MR data can be exploited to provide complementary information from adjacent slices with different sampling patterns. It enables highly partial Fourier imaging without losing image details or significant noise amplification. Also, the present invention allows the use of deep learning for the reconstruction of multi-slice partial Fourier MRI images with different sampling patterns.

The invention jointly uses multiple consecutive slices for reconstruction with a residual neural network (ResNet), which can be further enhanced by sampling adjacent slices in a complementary manner. The method can fully exploit structural and phase similarity in adjacent slices to synthesize missing k-space (spatial frequency) data.

In a third embodiment the present invention utilizes a deep learning (DL)-based joint reconstruction method for single-channel multi-contrast MR data with a uniform orthogonal under sampling pattern across different contrasts. It enables exploitation of the rich structural similarities from multiple contrasts as well as the incoherency that arises from complementary sampling.

The method comprises acquiring complex MRI image data as training data; training reconstruction models to predict complex MRI image data from highly under sampled data; and applying trained models to reconstruct unseen complex MRI image data from under sampled data. This aims to accelerate the reconstruction of multi-contrast MR images whose reconstruction is inherently slow. The results show that the proposed method can achieve robust reconstruction for single-channel multi-contrast MR data at R=4

According to a fourth embodiment the present invention performs an adaptive multi-contrast MR image denoising utilizing a deep learning (DL) strategy on flexible noise levels using a residual convolutional network architecture (U-Net) with a noise level map. The introduced noise level map can be manually set to fit different noise levels. This method utilizes the structural similarities across contrasts by simultaneously denoising multiple contrasts. The denoising results outperform BM3D in terms of noise reduction and detail preservation. More importantly, a noise-level map can be manually set to fit the different noise levels.

The adaptive DL-based strategy can be applied simultaneously to denoise multi-contrast MR images with different noise levels using only a single model, which is implemented by combining U-Net architecture [21] with ResNet architecture [22].

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing and other objects and advantages of the present invention will become more apparent when considered in connection with the following detailed description and appended drawings in which like designations denote like elements in the various views, and wherein:

FIG. 1 is a diagram of the joint denoising method of the present invention;

FIGS. 2A and 2B show the results of denoising according to the present method, where FIG. 2A shows denoising results of the simulated DW images and FIG. 2B shows resulting diffusion metric maps computed from denoised DW images, where the image set contains one b0 and 6 DW images with b=1000 s/mm2, but only one b0 image and one DW image along one direction are shown in FIG. 2A;

FIGS. 3A and 3B illustrate denoising results with FIG. 3A) showing vivo DW brain images and FIG. 3B showing the resulting diffusion metric maps computed from denoised DW images;

FIG. 4 shows the block matching results of the in vivo DW images in FIG. 3A;

FIG. 5A shows the denoising results with in vivo DW brain images and FIG. 5B shows the resulting diffusion metric maps computed from denoised DW images;

FIG. 6 is a flowchart of the EMS-PF reconstruction method according to the present invention;

FIG. 7A is a series of photographs showing reconstruction of conventional POCS and proposed SS-PF/MS-PF/EMS-PF for highly partial Fourier imaging (PF fraction=51%) and FIG. 7B shows the corresponding zoomed in view in the box in the photos of FIG. 5A;

FIG. 8 shows error maps of the reconstructed images in FIG. 7A with enhanced brightness (×10) and corresponding peak signal-to-noise ratio (PSNR)/structural similarity (SSIM) at PF fraction=0.51;

FIG. 9 is a comparison between the EMS-PF reconstruction according to the present invention and the conventional POCS method for varying PF factors (0.51, 0.55, and 0.65), as well as the error maps (×10);

FIG. 10A is a photographic evaluation of T2-weighted fast spin echo and FIG. 10B is an evaluation of T1-weighted gradient echo brain images with highly partial Fourier imaging (PF fraction=0.51);

FIG. 11 is a diagram of the method according to the present invention using a Res-UNet architecture;

FIG. 12 illustrates multi-contrast reconstruction for multi-contrast MR;

FIG. 13 shows the results for MR data containing a small lesion (indicated by red rectangular) with a uniform under sampling factor;

FIG. 14 illustrates multi-contrast reconstruction for multi-contrast MR data with aliasing artifacts removed and structural details preserved;

FIG. 15A is a block diagram showing the architecture of the multi-contrast denoising method of the present invention, FIG. 15B illustrates the residual blocks of the present invention, which consist of two convolutional layers with a ReLU activation in between, FIG. 15C shows the Strided conv2D block, which consists of a convolutional layer and ReLU activation and FIG. 15D shows the transposed conv2D block, which consists of a transposed convolutional layer and ReLU activation (d);

FIG. 16 presents MRI slides showing the results of using the multi-contrast denoising method of the present invention with the same level of noise (noise standard deviation σ=15) added to simulate noisy images;

FIG. 17 shows MRI slides with the denoising results for images with a higher noise level, i.e., where the same level of noise (σ=25) was added to form noisy images;

FIG. 18 shows the denoising results of the present invention with different noise levels for different contrasts (σ=5, 15, and 25 for T1w, T2w, and FLAIR, respectively), as well as non-optimal results to show the effect of adjusting the noise level map; and

FIG. 19 shows the denoising results for clinical images with pathology.

### DETAILED DESCRIPTION OF THE INVENTION

### Embodiment #1—Joint Denoising of Diffuse-Weighted Images Using a Weighted Nuclear Norm Minimization Approach

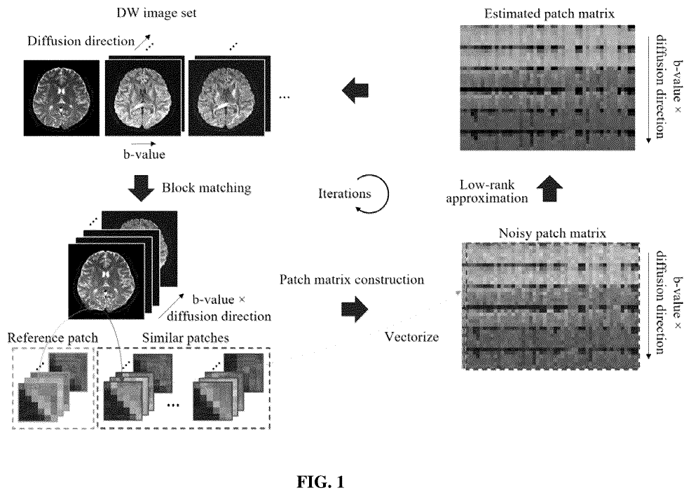

FIG. 1 is a diagram of the joint denoising method of the present invention. Within each iteration the method comprises the steps of: (1) extracting reference patches using a sliding window and searching for similar patches through block matching; (2) for each reference patch, stretching similar patches to vectors and stacking them into a matrix to form a low-rank patch matrix; (3) for each patch matrix, estimating a noise-free patch matrix through a weighted nuclear norm minimization (WNNM) model; and (4) converting estimated patch matrices back to images.

As shown in FIG. 1 the Block matching is achieved by sliding a 3D window across the entire DW image set. Then the 3D reference patches are extracted. For each reference patch, k similar patches are searched based on Euclidean distance.

Next Patch matrix construction is achieved by stretching k similar patches to vectors and stacking them into a matrix. The patch matrix is then multiplied by a weighting matrix, which is a diagonal matrix, and determined by the noise level of each image.

The Low-rank approximation is carried out with a weighted nuclear norm minimization (WNNM) model [7] [8], which is applied for estimating noise-free patch matrices. For each noisy patch matrix, an estimated patch matrix can be obtained by performing matrix singular value decomposition to the patch matrix, and singular-value thresholding of the singular matrix.

Finally, the Recovering DW image is set from the estimated patch matrices.

The method can be further improved by:
1) Using complex-valued images as an input so that the method will deal with Gaussian distributed noise;
2) Using a patch-based noise estimation method so that the method can be used for denoising spatially varied noise.
3) Using other criteria for block matching (e.g. SSIM or photometric distance) so that structural similarities can be better explored.

A simulation experiment was carried out to prove the invention. In particular, simulated brain data was originally created for ISMRM 2015 Tractography challenge. [9] The dataset contains one b=0 image and 32 DW images with b-value=1000 s/mm². The matrix size is 90×108×90 with 2-mm isotropic resolution. One b=0 image and 6 DW images were extracted to form a ground truth image set to evaluate the proposed denoising method for simple DTI. Rician noise (4% of the maximum intensity) was added to form noisy DW images.

In an in vivo Experiment two brain datasets were acquired on a 3T Philips scanner using an 8-channel coil by 4-shot interleaved EPI with matrix size=220×220, slice number=10. The imaging parameters for the first dataset were TR/TE=2400/118 ms, 6 diffusion directions with b=2000 s/mm2 and a b=0 image. The scan was repeated 10 times for a high SNR reference. The imaging parameters for the second dataset were TR/TE=2500/123 ms, 6 diffusion directions with b=1000/2000/3000 s/mm2 and a b=0 image. The scan was repeated 4 times. For both datasets, the DW images of a single average were used as noisy image sets for denoising.

The denoising was also performed through Mixtures of Probabilistic Principal Component Analysers (MPPCA) [10], [11], [12] for comparison. Variance stabilizing transformation (VST) [13] and inverse VST were performed on the noisy image sets before and after denoising, respectively, so that the Rician noise could be treated as noise with unitary variance. For the denoising method, the sliding window size was 4×4 and similar patch number was k=140. The FSL DTIFit Toolbox [14] was used to derive quantitative diffusion maps. In the simulation experiment, the error maps were calculated by subtracting denoised images from ground truth images and measuring the normalized root-mean-square errors (NRMSE).

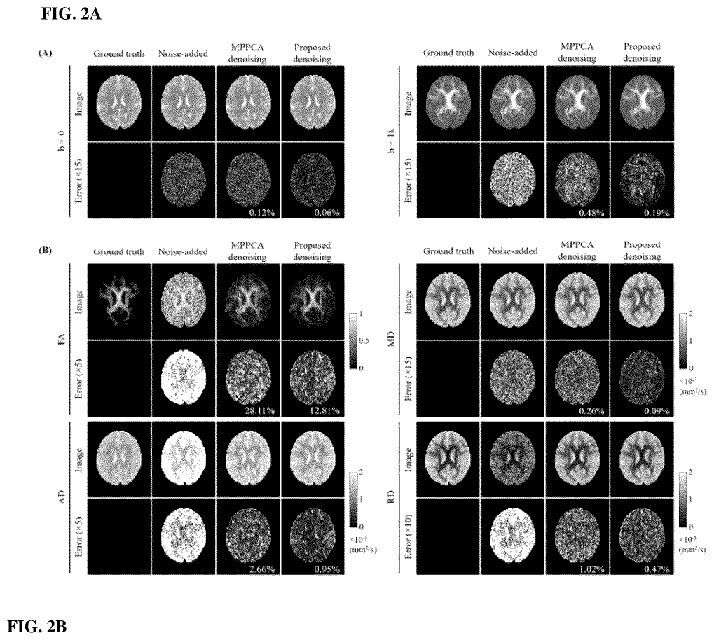

FIGS. 2A to-5B show the denoising performance of the present method. FIGS. 2A and 2B present denoising results for simulated images. The method effectively reduced noise in all DW images while preserving structural details, resulting in smaller normalized root-mean-square error (NRMSEs) than (MPPCA) for denoised images and quantitative diffusion maps.

FIG. 2A shows the results of the proposed method with simulated DW images and FIG. 2B shows the resulting diffusion metric maps computed from denoised DW images. The image set contains one b0 and 6 DW images with b=1000 s/mm2. For simplicity, only one b) image and one DW image along one direction are shown. Rician noise (4% of the maximum intensity) was added to ground truth images to obtain noisy images. The method of the present invention effectively reduced noise in all DW images and improved the estimation of diffusion metric maps in terms of revealing microstructural details in a fractional anisotropy (FA) map and achieving smaller NRMSEs compared with MPPCA method.

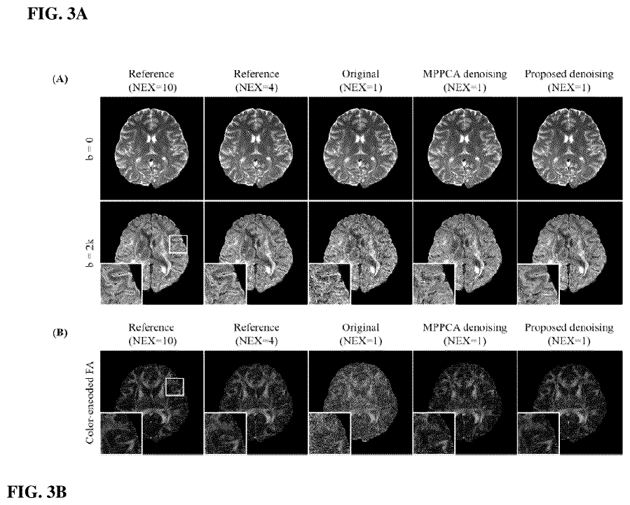

FIG. 3A shows the denoising results for an in vivo brain image set containing one b0 image and 6 DW images. This figure shows that when the DW images had low SNR, MPPCA became less effective, while the present method reduced the noise significantly and achieved image quality and FA map comparable to those using 4 averages.

The denoising results with the in vivo DW brain images are shown in FIG. 3A and resulting diffusion metric maps computed from denoised DW images are shown in FIG. 3B. The image set contains one b0 image and 6 DW images with b=2000 s/mm2. Only DW images along one direction are shown. The image set of NEX=1 was used for denoising, while the image set of NEX=4/10 was used as a high SNR reference. At very low SNR, the method was still robust and more effective than MPPCA in reducing noise while recovering structural details when compared to the reference. It achieved image quality and an FA map comparable to those using 4 averages.

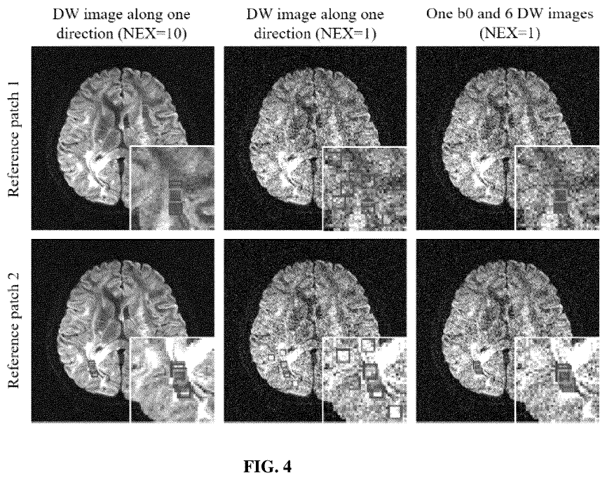

FIG. 4 shows block matching results of the in vivo DW images in FIG. 3. The block matching results for two reference patches are compared to those using a single DW image with NEX=10 and NEX=1. Note that at low SNR, the block matching results could be biased by noise. However, using the whole DW image set achieved block matching results similar to those using an image with high SNR (NEX=10), indicating that the structural similarities among DW images substantially improved the robustness of block matching, resulting in more similar patches and more rank-deficient patch matrices.

Note that at low SNR, the method still maintained high accuracy in searching similar patches by exploiting the structural similarities among DW images.

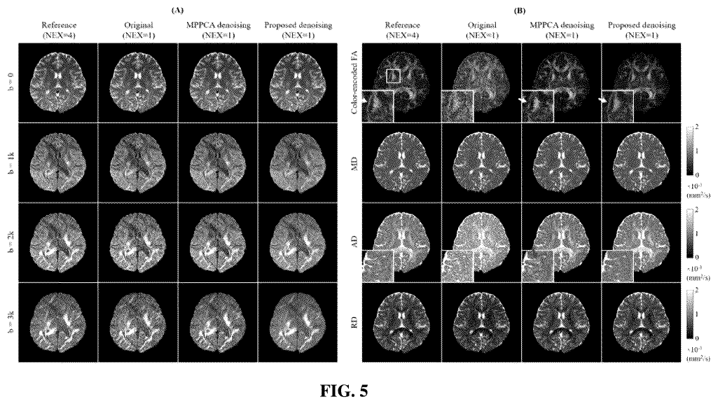

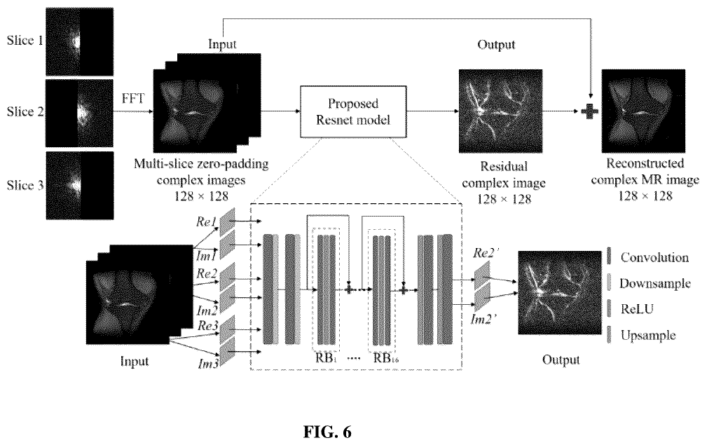

FIG. 5A presents denoising results with the in vivo DW brain images and FIG. 5B shows the resulting diffusion metric maps computed from denoised DW images. The image set contains one b0 image and 6 DW images with three b values, b=1000/2000/3000 s/mm2. Only DW images along one direction are shown. The image set of NEX=1 was used for denoising. Note that residual noise in MPPCA denoised images caused artifacts in the AD map, while the present method achieved metric maps comparable to those using 4 averages. The results clearly demonstrated that the method effectively reduced noise when DW images had different noise levels, and with FA revealing more microstructural details.

In summary, this new method for jointly denoising DW images exploits structural similarities of diffusion-weighted images, yielding significant noise reduction in all images and revealing more microstructural details. The superior performance of the method is based on the rationale that similar patches from noisy images can be extracted and used to form a patch matrix, which should be a low-rank matrix and thus can be recovered through low-rank matrix approximation. Further the method can be carried out in three directions. First, the method can be extended to jointly denoise multi-slice DW images. By concatenating multi-slice patch matrices, a low-rank patch tensor can be obtained and high-order singular value decomposition can be performed for the low-rank tensor approximation. Second, the noise level estimation can be optimized in a patch-based way so that the method can be more robust for non-uniformly distributed noise. Third, the method can be used for advanced diffusion MRI techniques, such as Tractography, Q-ball imaging and kurtosis imaging.

### Embodiment #2—Enhancing Multi-Slice Partial Fourier MRI Reconstruction Using Residual a Network

The present invention utilizes deep learning for partial Fourier (PF) reconstruction, which is applied to individual MRI slices and is termed “single-slice partial Fourier reconstruction” (SS-PF) [5]. With the present invention, multiple partial-Fourier acquired slices are jointly used for reconstruction. Multi-slice partial Fourier (MS-PF) reconstruction can be further enhanced by sampling adjacent slices in a complementary manner, termed “enhanced multi-slice partial Fourier” (EMS-PF) reconstruction. In brief, odd/even slices representing opposite halves of k-space are sampled for either readout or phase-encoding directions. FIG. 6 illustrates the flowchart of the proposed EMS-PF method, where three consecutive slices (Slices 1-3) are jointly used for reconstructing the central slice (Slice 2). The Input of the network has 6 concatenated channels for the real Re and imaginary Im parts of images from the three consecutive slices having complementary sampling patterns. The Output has 2 channels for the real Re2′ and imaginary parts Im2′ of the estimated residual image for the central slice. After that, the final reconstructed image is obtained by adding the residual image to the image reconstructed from zero-padding k-space. The upper part of FIG. 6 is the overall view, while the lower part shows the details from the Input through the Resnet model to the Output and the Reconstructed complex MR image.

In the lower part of FIG. 6 the real and imaginary parts for Slice 1 are Re1, Im1 for Slice 2 they are Re2, Im2 and for Slice 3 they are Re3, Im3. For the output Re2′ and Im2′ denote the real and imaginary parts of the output predicted residual image. The ResNet model has 16 radial bases (RBs) and each of them contains 2 convolutional layers followed by a Rectified Linear Unit (ReLU) activation function. In each convolutional layer, 64 convolutional kernels of size 3×3 are included.

Knee datasets from the Center for Advanced Imaging Innovation and Research (CAI2R) [6] were used for training, validating, and testing in the network. The coronal proton density weighted knee data were acquired using 2D fast spin echo (FSE), with TR=2200-3000 ms, TE=27-34 ms, FOV=160×160 mm2, matrix size=320×320, slice thickness/gap=3/0 mm. The data were acquired using a 15-channel knee coil, but combined to approximate single-channel acquisition. The datasets contained 942 subjects (each has 16 slices), 70%, 15%, and 15% of which were used for training, validation, and testing, respectively. The raw k-space was cropped to 128×128, retrospectively under sampled along a phase-encoding direction at PF fraction of 51%, 55%, and 65%. At PF fraction=51%, with only 4 symmetrically central k-space lines, it would be extremely challenging for conventional projection-onto-convex-sets (POCS) reconstruction.

Models for reconstructing the odd/even slices were trained separately, as their sampling patterns differed from each other. It took ˜3 hours to train each model with 100 epochs. The performance was quantitatively evaluated by the peak signal-to-noise ratio (PSNR) and structural similarity (SSIM).

Although trained with knee data only, the method was also evaluated with human brain datasets acquired on a 3T Philips MRI scanner using a single-channel head coil. T2-weighted images were acquired using 3D FSE with TR/TE=2500/213 ms, FOV=240×240×120 mm3, matrix size=240×240×120. T1-weighted images were acquired using 3D GRE with TR/TE=19/4 ms, flip angle=30°, FOV=240×240×130 mm3, matrix size=240×240×130. A 1D inverse Fourier transform was applied to the raw k-space, generating 2D k-space for multiple consecutive axial slices with slice thickness/gap=1/0 mm. The generated 2D k-space was cropped to 128×128 to fit the trained models, retrospectively under sampled along a phase-encoding direction.

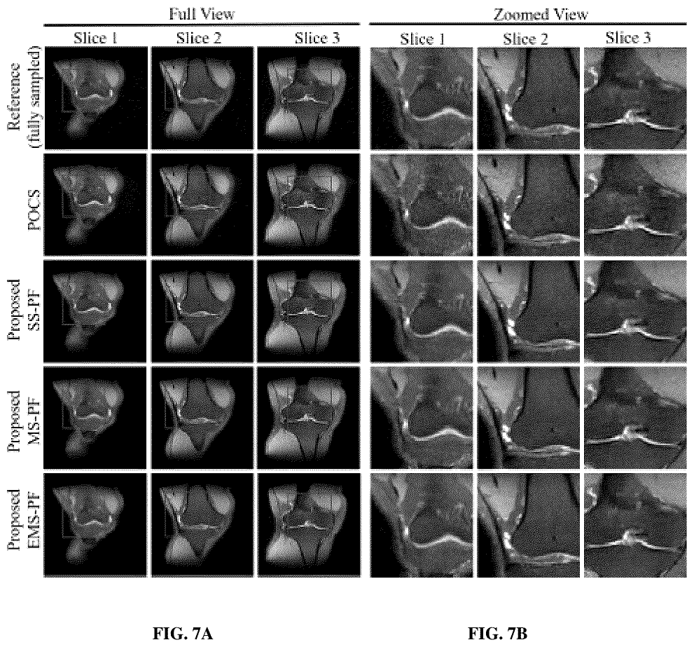

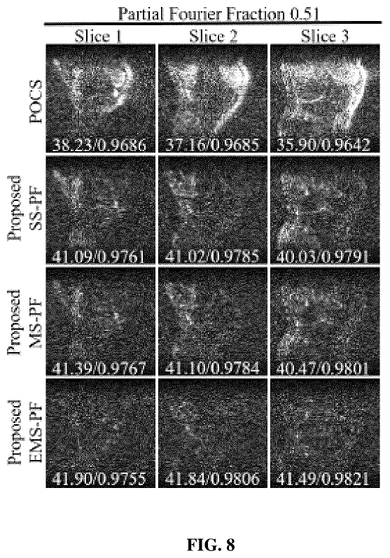

FIGS. 7A & B and 8 compare the reconstruction results and corresponding error maps for the conventional POCS and the SS-PF/MS-PF/EMS-PF methods of the present invention. In particular, FIG. 7A shows reconstruction of conventional POCS and the SS-PF/MS-PF/EMS-PF methods for highly partial Fourier imaging (PF fraction=51%). The corresponding zoomed view is shown on the right in FIG. 7B. Images details indicated by the red arrows were best preserved in the EMS-PF results. Note that at PF fraction=51%, the performance of conventional POCS reconstruction degraded dramatically due to insufficient central k-space data for phase estimation. The EMS-PF method of the present invention showed the best performance on recovering image details without noise amplification among the aforementioned approaches. The MS-PF method and SS-PF method had similar performance, which suggested that without complementary sampling across adjacent slices, jointly using multiple slices for reconstruction cannot fully exploit their structural and phase similarities. FIG. 8 shows the error maps of the reconstructed images in FIGS. 7A & B with enhanced brightness (×10) and corresponding peak signal-to-noise ratio (PSNR)/structural similarity (SSIM) at PF fraction=0.51. From these views it is obvious that the EMS-PF method of the present invention outperforms the other methods in terms of reduced residual error.

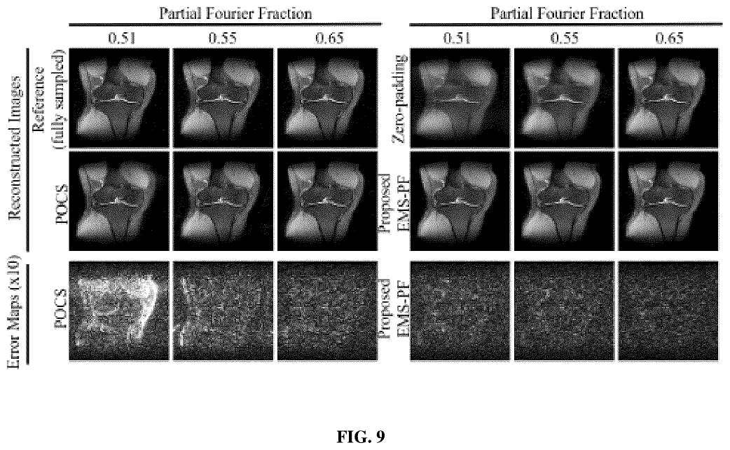

FIG. 9 is a comparison between the EMS-PF reconstruction of the present invention and the conventional POCS method for varying PF factors (0.51, 0.55, and 0.65). With a larger portion of data omitted, the EMS-PF method suffered a slightly higher residual error, while the performance of the POCS method substantially deteriorated. The EMS-PF method yields a slightly higher error as the PF fraction decreases. This demonstrates the EMS-PF enables highly partial Fourier imaging.

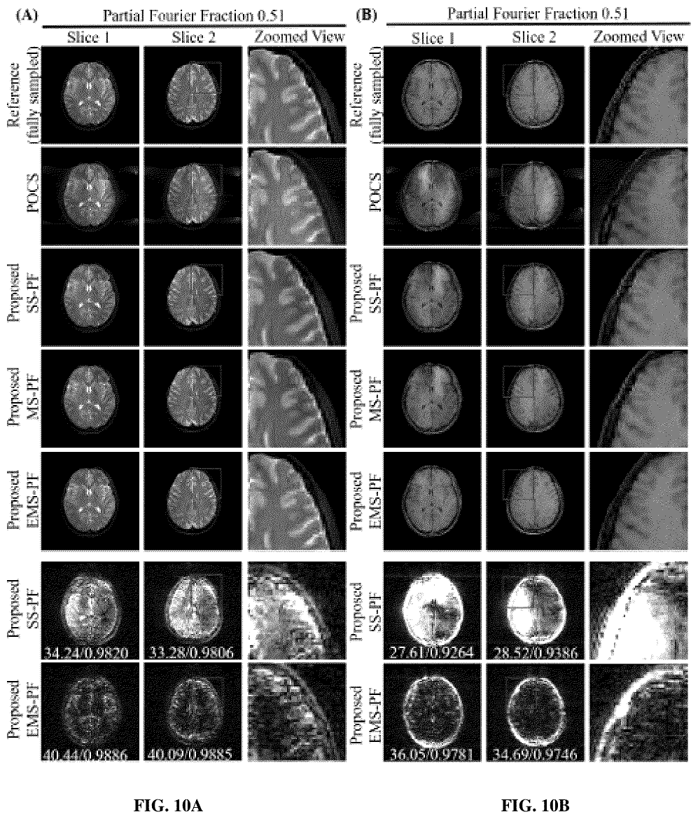

FIG. 10 presents reconstructed image from T1-weighted/T2-weighted brain images, demonstrating the robustness of the EMS-PF method for MR data of different anatomical regions and/or acquired with different sequences. FIG. 10A is an evaluation with T2-weighted fast spin echo and FIG. 10B is an evaluation with T1-weighted gradient echo brain images with highly partial Fourier imaging (PF fraction=0.51). The EMS-PF method had substantially reduced residual error compared to the other methods. Error maps (brightness ×10) and quantitative assessment (PSNR/SSIM) in the last two rows of FIGS. 10A and 10B suggested that the present EMS-PF approach outperformed the SS-PF method.

The method of the present invention exploits the structural and phase similarity in adjacent slices to synthesize the missing k-space in the slice to be reconstructed, which is superior in preserving the image details without amplifying noise, especially for highly partial Fourier imaging. Note that the slice thickness/gap will affect the performance of the proposed approach as increased slice thickness/gap decreases similarities of adjacent slices, which can be considered by adjusting the number of jointly used slices. The proposed approach may also be used for 2D partial Fourier imaging, multi-channel MR acquisition, and/or integrated with parallel imaging.

### Embodiment #3—Multi-Contrast MRI Reconstruction from Single-Channel Uniformly Under Sampled Data

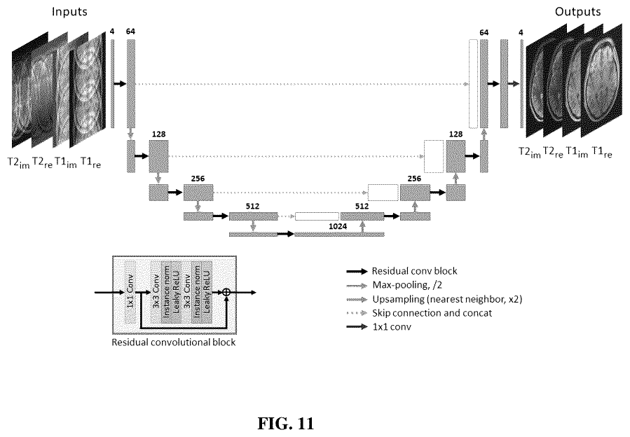

According to the invention a 2D Residual U-Net (Res-UNet) architecture, which consists of 4 pooling layers, is implemented for jointly reconstructing MR data with orthogonal under sampling directions across different contrasts. The Res-UNet, as shown in FIG. 11, is a U-Net framework with residual convolutional blocks. Real and imaginary components of complex T1- and T2-weighted images (T2im, T2re, T1im, T1re) are input to the model as separate channels. The network is trained using an Adam optimizer with initial learning rate 10-4, decay factor of 0.1 per 10 epochs, and 12 loss function. The model is trained for 30 epochs on a single GTX1080Ti.

In implementing the invention 400 T1- and T2-weighted MR volumes from the HCP S1200 dataset [17] were used for model training, validation and testing. Multi-contrast MR data were prepared as follow:
1) co-registering the T1- and T2-weighted MR volumes subject-by-subject using FSL FLIRT [18],
2) down sampling the images by a factor of 2, resulting in identical in-plane geometry: FOV=224×180 mm² and resolution=1.4×1.4 mm²,
3) adding different synthetic random 2D nonlinear phases to T1- and T2-weighted MR volumes separately, and
4) applying orthogonal 1D uniform under sampling. The dataset was randomly split into training, validation and testing sets at a ratio of 8:1:1.

Experimental results were quantitatively evaluated using structural similarity index (SSIM) and normalized root mean square error (NRMSE) methods. The performance of Res-UNet with complementary k-space sampling was evaluated with 1D acceleration at R=3 and 4. The proposed method was also evaluated with images containing pathological regions.

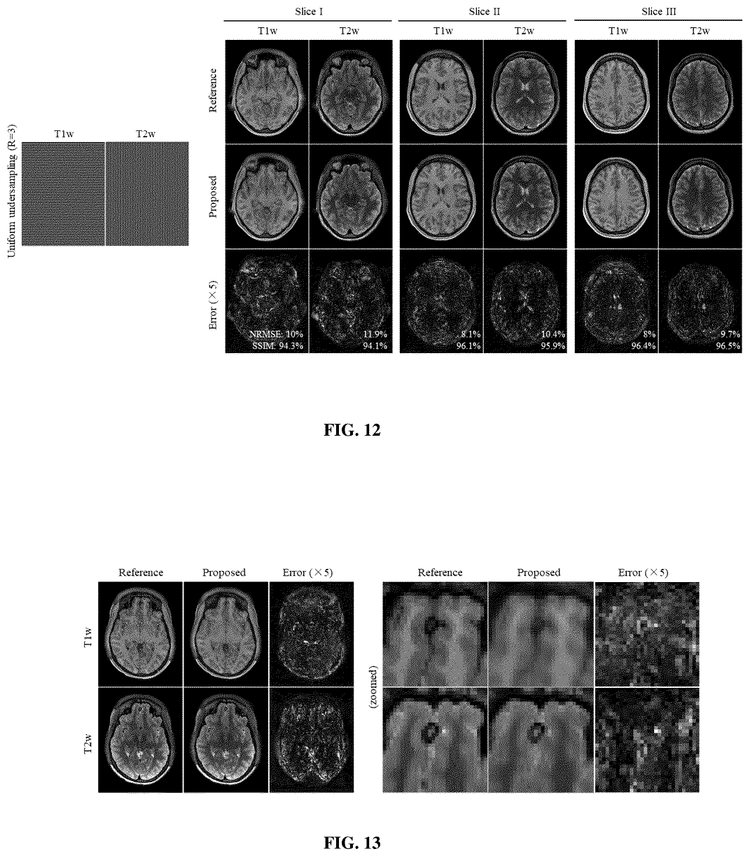

FIG. 12 shows reconstructed images obtained using the method of the present invention at R=3. The method successfully removed aliasing artifacts introduced by the uniform under sampling. Thus, FIG. 12 clearly demonstrates that the model of the present method can reconstruct high-fidelity images without obvious artifacts.

FIG. 13 shows results for MR data containing a small lesion (indicated by red rectangular) with uniform under sampling factor R=3. Note that the small lesion remained faithfully reproduced in the reconstructed images though the model in FIG. 11 was trained with normal subjects. Thus, FIG. 13 shows the robustness of the multi-contrast MR reconstruction of the present invention for brain images with pathology regions, where the correlations of different contrasts might differ from that in normal tissue.

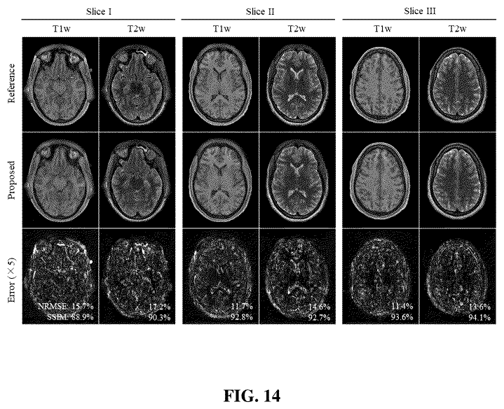

FIG. 14 illustrates multi-contrast reconstruction for multi-contrast MR data at R=4, which indicates the capability of the proposed model to remove aliasing artifacts and preserve structural details even at an under sampling rate of R=4. Thus, it shows that the present method can achieve an under sampling factor of 4 with two contrasts jointly reconstructed.

The present invention utilizes a DL-based reconstruction for multi-contrast MR data, and its effectiveness is demonstrated on a single-channel MR dataset with T1w/T2w contrasts. The results indicated that the method can effectively remove aliasing artifacts at R=3.

The results on pathological brain tissue showed that the method, which was not specifically trained on a pathology dataset, can reasonably reconstruct the pathology. Orthogonally alternating the PE direction increases the incoherency of aliasing caused by uniform under sampling. This incoherency allows similar reconstruction results to 2D random under sampling. [19] Thus, joint reconstruction with orthogonal PE direction can also be realized via deep learning. Patch based loss (e.g. SSIM [20]) can be used instead of pixel-wise L2 loss, which can further improve the reconstruction in terms of reduced blurring effects.

### Embodiment #4—Adaptive Multi-Contrast MRI Denoising Based on Residual U-Net Using a Noise Level Map

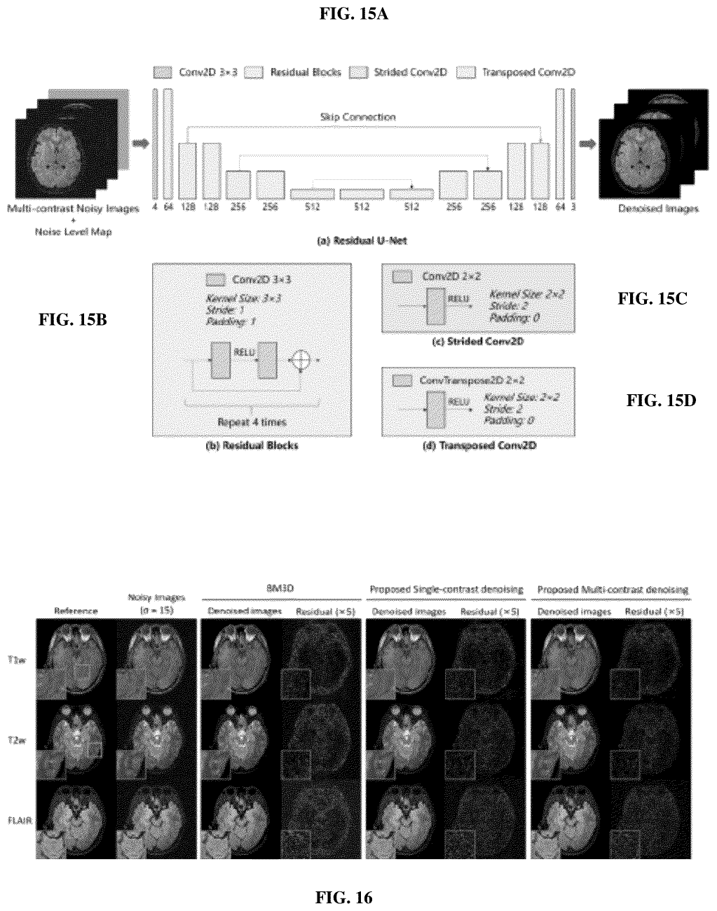

MRI denoising recovers high-quality MR images y from the noisy MR images x. Generally, the neural network seeks a mapping function f that minimizes the difference between the denoised images and target noise-free images. The method of the invention as shown in FIG. 15 uses a residual U-Net architecture, which combines 4-scale U-Net and ResNet. ReLU activations that are used after strided/transposed convolutional layers and between two convolutional layers within each residual block. All the convolutional layers are bias-free to impose scaling invariance.

FIG. 15A shows the architecture of the multi-contrast denoising method of the present invention. In evaluating the present invention multi-contrast noisy images plus added noise are applied to the residual U-Net system. The system is comprised of connected residual blocks (Conv2d 3×3), Strided Conv2d blocks and Transposed Corv2D blocks. The results of the process are denoised images. FIG. 15B illustrated the residual blocks, which consist of two convolutional layers with a ReLU activation in between. This is repeated 4 times. The Kernel Size is 3×3, the Stride is 1 and the Padding is 1. FIG. 15C shows the Strided conv2D block, which consists of a convolutional layer and ReLU activation. The Kernel Size is 2×2, the Stride is 2 and the Padding is 0. FIG. 15D shows the transposed conv2D block, which consists of a transposed convolutional layer and ReLU activation. The Kernel Size is 2×2, the Stride is 2 and the Padding is 0.

Images of different contrasts are input as different channels. Inspired by FFDNet [23] and DRUNet, [24] a noise level map is introduced as an additional input channel to balance noise reduction and preserve detail. The noise level map can be manually adjusted to fit the input noise level, which is considered to be uniform within the FOV. The denoised images are output as different channels.

The network parameters are adjusted by minimizing the L1 loss between the denoised images and their ground-truths with an Adam optimizer. A pre-trained model [24] is used for initialization. In testing the invention 5800 multi-contrast image sets were selected from the HCP dataset. T1-weighted (T1w) images were acquired with MPRAGE with TR/TE/TI=2400/2.1/1000 ms, flip angle (FA)=8°. T2-weighted (T2w) were acquired using 3D FSE with TR/TE=3200/565 ms. All images had an isotropic resolution of 0.7×0.7×0.7 mm3. Tiw image, T2w image, and the averaged of T1w/T2w images were treated as three different contrasts. Noisy images were generated by adding complex white Gaussian noise with standard deviation (a) ranging from 0 to 35 and used to train the proposed model.

The proposed method was also evaluated with human brain datasets acquired on a 3T Philips MRI scanner using a single-channel head coil. Tiw images were acquired using 3D gradient-echo (GRE) with TR/TE=19/4 ms, FA=30°, T2w images were acquired using 3D fast spin echo (FSE) with TR/TE=2500/213 ms. T2-weighted FLAIR images were acquired with 3D FSE with TR/TI/TE=4800/1650/282 ms. All images have an isotropic resolution of 1×1×1 mm3. Complex white Gaussian noise of different levels was added to the reconstructed complex images. After that, the magnitude images were used for evaluating the method.

FIG. 16 shows the results using the multi-contrast denoising method of the present invention. The same level of noise (noise standard deviation σ=15) was added to simulate noisy images. The method preserved the structural details while removing noise. These noisy images were also denoised using block-matching and 3D filtering (BM3D). However, the results of BM3D were over-smoothed and suffered from severe structural details loss

In FIG. 16, while the same level of noise was added to the three images they had different contrasts. The approach of the invention preserved the structure details with the noise significantly suppressed.

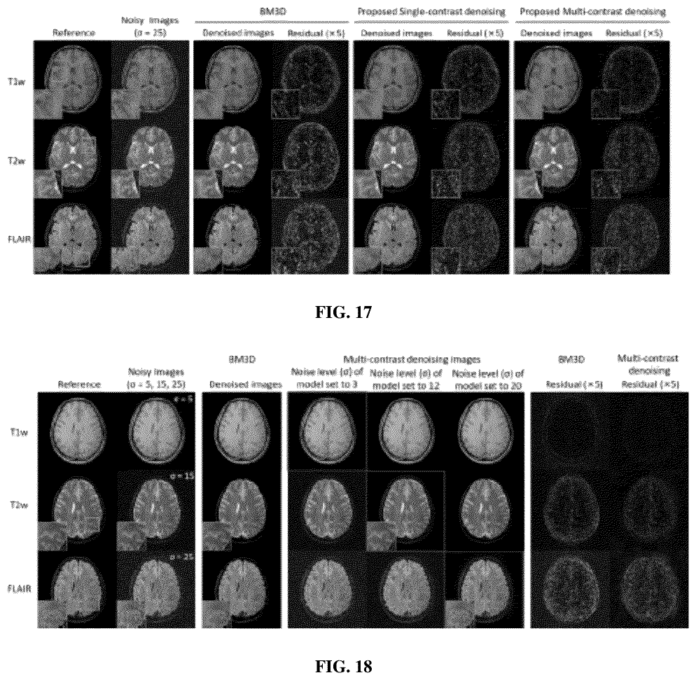

FIG. 17 shows the denoising results for images with a higher noise level than in FIG. 16. In FIG. 3A the same level of noise (σ=25) was added to form more noisy images, but the method maintained similar performance, while BM3D method smoothed the image details even more if an attempt was made to reduce the noise to the same level as the present method. FIG. 3A shows the robustness of the method even when the images are corrupted with a much higher noise level.

The denoising results with different noise levels for different contrasts (σ=5, 15, and 25 for T1w, T2w, and FLAIR, respectively) are shown in FIG. 18. Optimal noise level maps for each contrast were empirically determined to adapt the noise level of different contrasts. The red boxes indicate the best results of the method. Non-optimal results were also displayed to show the effect of adjusting the noise level map. Again, the present invention was able to effectively remove noise without losing structural details in the noisy T2w and FLAIR images, yielding better results than the BM3D method.

In particular FIG. 18 indicates that the methods still perform well in terms of noise reduction and detail preservation when images of different contrasts have different noise levels. In such a situation, the noise level map can be adjusted to adapt the noise level of each contrast, so to achieve the best performance for all contrasts.

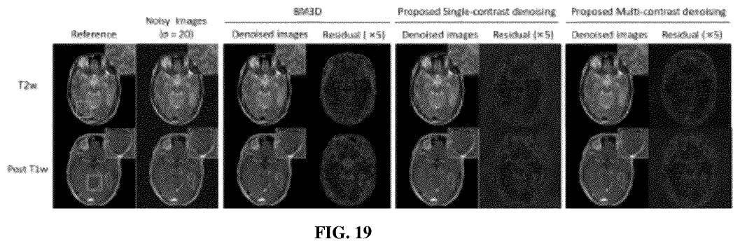

FIG. 19 shows the denoising results for clinical images with pathology. The same level of noise (σ=20) was added to form noisy images. The method did not compromise the structural details in the pathological regions that manifested decreased structural similarities. However, the BM3D method over-smoothed the structures, even in normal regions. Thus, FIG. 19 shows that even with pathology that manifested insufficient structural similarities across contrasts, the method could remove noise with structural details well preserved. In contrast, the BM3D method removed the same level of noise but over-smoothed the image structures.

In summary the denoising method of the present invention utilizes the structural similarities between MRI slices by simultaneously denoising multiple contrasts using a residual U-Net. It shows satisfactory performance in both noise reduction and details preservation at different noise levels. This is achieved because the noise level map can be manually adjusted to fit different noise levels. Further, in the presence of a slight geometrical mismatch across different contrasts, such as can occur with pathology, the method still works because the receptive field of the model is large enough that the extracted information can tolerate subtle geometrical mismatch. Note that images obtained with parallel imaging will have a spatially variant noise distribution. More importantly, such spatial variation can differ across different contrasts, as they may have non-identical sampling patterns. The method can be designed to have individual noise maps for each contrast to compensate for this issue.

The cited references in this application are incorporated herein by reference in their entirety and are as follows:
[1] Margosian, Paul M., DeMeester, Gordon, and Liu, Haiying. “Partial Fourier Acquisition in MRI.” Encyclopedia of Magnetic Resonance. Highland Heights: Picker International, 2007.
[2] Koopmans, Peter J., and Pfaffenrot, Viktor. “Enhanced POCS reconstruction for partial Fourier imaging in multi-echo and time-series acquisitions.” Magnetic Resonance in Medicine 85.1 (2020): 140-151.
[3] Liu, Yilong, et al. “Calibrationless parallel imaging reconstruction for multislice MR data using low-rank tensor completion.” Magnetic Resonance in Medicine 85.2 (2020): 897-911.
[4] Weizman, Lior, et al. “Exploiting similarity in adjacent slices for compressed sensing MRI.” 2014 36th Annual International Conference of the IEEE Engineering in Medicine and Biology Society. IEEE, 2014.
[5] Cao, Peipei, et al. “Partial Fourier MRI Reconstruction Using Convolutional Neural Networks.” Proceedings of International Society of Magnetic Resonance in Medicine. 2020.
[6] Knoll, Florian, et al. “Welcome to the fastMRI Dataset.” NYU Langone Health. https://fastmri.med.nyu.edu/. Accessed 14 Oct. 2020.
[7] Y. Zhao, Y. Liu, H. K. Mak, and E. X. Wu. Simultaneous denoising of multi-contrast MR images using a novel weighted nuclear norm minimization approach. 27th Annual Meeting of ISMRM, No. 0669, 2019.
[8] J. Xu, L. Zhang, D. Zhang, and X. Feng. Multi-channel weighted nuclear norm minimization for real color image denoising. IEEE International Conference on Computer Vision, 2017.
[9] P. F. Neher, F. B. Laun, B. Stieltjes, and K. H. Maier-Hein. Fiberfox: facilitating the creation of realistic white matter software phantoms. Magn Reson Med 2014; 72(5): 1460-1470.
[10] J. Veraart, E. Fieremans, and D. S. Novikov. Diffusion MRI noise mapping using random matrix theory. Magn Reson Med 2016; 76(5): 1582-1593.
[11] J. Veraart, D. S. Novikov, D. Christiaens, B. Ades-aron, J. Sijbers and E. Fieremans. Denoising of diffusion MRI using random matrix theory. NeuroImage 2016; 142: 394-406.
[12] X. Ma, K. Ugurbil, and X. Wu. Denoise magnitude diffusion magnetic resonance images via variance-stabilizing transformation and optimal singular-value manipulation. NeuroImage 2020; 215: 116852.
[13] A. Foi. Noise estimation and removal in MR imaging: The variance-stabilization approach. IEEE International symposium on biomedical imaging. IEEE, 2011.
[14] M. Jenkinson, C. F. Beckmann, T. E. Behrens, M. W. Woolrich, and S. M. Smith. Fsl. Neuroimage 2012; 62(2): 782-790.
[15] Polak D, Cauley S, Bilgic B, et al. Joint multi-contrast variational network reconstruction (jVN) with application to rapid 2D and 3D imaging. Magn Reson Med. 2020; 84:1456-1469.
[16] Yi Z, Liu Y, Zhao Y, Chen F, Wu EX. Joint calibrationless reconstruction of highly undersampled multi-contrast MR datasets using a novel low-rank completion approach. In 27th Annual Meeting of ISMRM 2019. Montreal.
[17] Van Essen, D. C., et al: The wu-minn human connectome project: an overview. Neuroimage 80, 62-79, 2013.
[18] M. Jenkinson and S. Smith, “A global optimisation method for robust affine registration of brain images,” Med. Image Anal., 2001.
[19] Wang H, Liang D, Ying L. Pseudo 2D random sampling for compressed sensing MRI. In: Conf Proc IEEE Eng Med Biol Soc, 2009, IEEE, 2672-2675.
[20] Hammernik K, Knoll F, Sodickson D K, Pock T. L2 or not L2: Impact of Loss Function Design for Deep Learning MRI Reconstruction. In Proceedings of the International Society of Magnetic Resonance in Medicine (ISMRM). 2017. 0687
[21] Ronneberger, Olaf, Philipp Fischer, and Thomas Brox. “U-net: Convolutional networks for biomedical image segmentation.” International Conference on Medical image computing and computer-assisted intervention. Springer, Cham, 2015.
[22] Kaiming He, Xiangyu Zhang, Shaoqing Ren, et al. “Deep residual learning for image recognition.” Proceedings of the IEEE conference on computer vision and pattern recognition. 2016.
[23] Kai Zhang, Wangmeng Zuo, Lei Zhang, et al. “FFDNet: Toward a fast and flexible solution for CNN-based image denoising.” IEEE Transactions on Image Processing 27.9 (2018): 4608-4622.
[24] Kai Zhang, Yawei Li, Wangmeng Zuo, et al. “Plug-and-play image restoration with deep denoiser prior.” arXiv preprint arXiv:2008.13751 (2020).
[25] Kostadin Dabov, Alessandro Foi, Vladimir Katkovnik, et al. “Image denoising by sparse 3-D transform-domain collaborative filtering.” IEEE Transactions on image processing 16.8 (2007): 2080-2095.

While the present invention has been particularly shown and described with reference to preferred embodiments thereof it will be understood by those skilled in the art that various changes in form and details may be made therein without departing from the spirit and scope of the invention, and that the embodiments are merely illustrative of the invention, which is limited only by the appended claims. In particular, the foregoing detailed description illustrates the invention by way of example and not by way of limitation. The description enables one skilled in the art to make and use the present invention, and describes several embodiments, adaptations, variations, and method of uses of the present invention.

What is claimed is:

1. A low-rank based method for jointly denoising diffusion weighted (DW) magnetic resonance imaging (MRI) images, comprising the steps of:
extracting reference patches using a sliding window and searching for similar patches through block matching; for each reference patch;
stretching its similar patches to vectors;
stacking the vectors m into a matrix to form a low-rank patch matrix;
estimating for each patch matrix a noise-free patch matrix through a weighted nuclear norm minimization (WNNM) model; and
converting estimated patch matrices back to images.

2. The method of claim 1 further including the step of multiplying the patch matrix by a weighting matrix, which is a diagonal matrix determined by a noise level of each image.

3. The method of claim 1 further including the steps of
using complex-valued images as an input so that the method will deal with Gaussian distributed noise;
using a patch-based noise estimation method so that the method can be used for denoising spatially varied noise; and
using other criteria for block matching (e.g.: SSIM or photometric distance) so that structural similarities can be better explored.

4. The method of claim 3 further including the steps of performing variance stabilizing transformation (VST) and inverse VST on the noisy image sets before and after denoising, respectively, so that Rician noise is treated as noise with unitary variance.

5. The method of claim 1 wherein by concatenating multi-slice patch matrices, a low-rank patch vector can be obtained and high-order singular value decomposition can be performed for the low-rank tensor approximation.

6. A method for reconstructing multi-contrast magnetic resonance imaging from single-channel uniformly under sampled data, comprising the steps of:
acquiring complex MRI image data as training data;
training reconstruction models to predict complex MRI image data from highly under sampled data; and
applying trained models to reconstruct unseen complex MRI image data from the under sampled data.

7. A method for reconstructing multiple partial Fourier MRI slices, comprising the steps of:
jointly acquiring real and imaginary parts of at least three partial-Fourier acquired slices having complementary sampling patterns;
using a deep learning algorithm to generate real and imaginary parts of two channels representing an estimated residual image of the central slice; and
adding the residual acquired image to the estimated residual image to form a reconstructed complex image.

8. A 2D Residual U-Net (Res-UNet) architecture for jointly reconstructing multi-contrast MR data with orthogonal under sampling directions across different contrasts, comprising:
four pooling layers with residual convolutional blocks;
separate channels for receiving real and imaginary components of complex T1- and T2-weighted images (T2im, T2re, T1im, T1re);
means for max pooling/down sampling between the layers from a first to a fourth layer;
means for up-sampling between the layers from the fourth to the first layer; and
means for performing a 1×1 conversion to provide the output.

9. The 2D Residual U-Net (Res-UNet) architecture of claim 8 wherein the network is trained using an Adam optimizer.

10. A system for multi-contrast MRI image denoising comprising a residual U-Net architecture, which combines 4-scale U-Net and ResNet. ReLU activations that are used after strided/transposed convolutional layers and between two convolutional layers within each residual block.

11. The system for multi-contrast denoising of claim 10 wherein the architecture is formed from connected residual blocks (Conv2d 3×3), Strided Conv2d blocks and Transposed Corv2D blocks.

12. The system for multi-contrast denoising of claim 11 wherein the Strided conv2D block comprises a convolutional layer and ReLU activation.

13. The system for multi-contrast denoising of claim 11 wherein the transposed conv2D block comprises a transposed convolutional layer and ReLU activation.

14. The system for multi-contrast denoising of claim 11 wherein images of different contrasts are input as different channels and further including a noise level map introduced as an additional input channel to balance noise reduction and detail preservation.

15. A method for denoising of multi-contrast MRI images utilizing the structural similarities between MRI slices by simultaneously denoising multiple contrasts using a residual U-Net.
